(19) United States
(12) Patent Application Publication
(10) Pub. No.: US 2014/0358003 A1
(43) Pub. Date: Dec. 4, 2014

(54) DIAGNOSTIC ULTRASOUND APPARATUS

(71) Applicant: HITACHI ALOKA MEDICAL, LTD., Mitaka-shi, Tokyo (JP)

(72) Inventors: Mitsuru Ueda, Mitaka-shi (JP); Takaya Uno, Mitaka-shi (JP)

(73) Assignee: HITACHI ALOKA MEDICAL, LTD., Mitaka-shi, Tokyo (JP)

(21) Appl. No.: 14/368,172

(22) PCT Filed: Nov. 15, 2012
(86) PCT No.: PCT/JP2012/079637

§ 371 Date: Jun. 23, 2014

(30) Foreign Application Priority Data
Jan. 11, 2012 (JP) 2012-002796

(51) Int. Cl. A61B 8/08; A61B 8/14

(52) U.S. Cl. CPC A61B 8/5253; A61B 8/14; A61B 8/5207
USPC 600/443

(57) ABSTRACT

In the diagnostic ultrasound apparatus, moving average processing is successively performed on a frame series with frame sets obtained from three kinds of frames, A, B, and C, as units, thereby successively generating compound frames. Each compound frame has a central sub-region and multiple peripheral sub-regions. In order to match the gain between the multiple sub-regions when generating each compound frame, a special compounding condition is applied to individual peripheral sub-regions. Specifically, processing that redundantly substitutes the same data value for multiple elements in the compounding computation equation is performed. Using such a spatial compounding, differences in gain between multiple sub-regions can be eliminated or reduced.

### TECHNICAL FIELD

The present invention relates to an ultrasound diagnostic apparatus, and in particular to a compounding process of a plurality of sets of frame data; that is, a spatial compounding process.

### BACKGROUND ART

Ultrasound diagnostic apparatuses are being used in the medical field. An ultrasound diagnostic apparatus transmits and receives ultrasound to and from a living body, and forms an ultrasound image based on information obtained by the transmission and reception of the ultrasound. When, for example, a carotid artery, a mammary gland, or the like is observed with a B-mode image (two-dimensional black-and-white tomographic image) using the ultrasound diagnostic apparatus, an electric linear scanning probe is used. In the electric linear scan, a transmission and reception opening for forming a beam is set on an array transducer in the probe, and the transmission and reception opening is scanned. With such a process, the ultrasound beam is linearly scanned, and a beam scanning plane is formed. Normally, the ultrasound beam is formed in a direction perpendicular to the array transducer; that is, a direction of a beam deflection angle of 0 degree. When the linear scanning of the ultrasound beam is executed under such a condition, a rectangular beam scanning plane would be formed. When a convex-type probe is employed, a beam scanning plane of a fan shape would be formed under the condition of the beam deflection angle of 0 degree.

In relation to the ultrasound diagnosis, in the related art, a spatial compounding method is known. In the spatial compounding, a plurality of beam scanning planes are repeatedly formed while switching the beam deflection condition in a circulating manner, and, thus, a frame data array arranged in time sequential order is obtained. In the frame data array, a compounding process is sequentially executed with a predetermined number of frames as a unit, to sequentially generate compounded frame data. According to the spatial compounding, an image can be formed based on data obtained by executing transmission and reception of the ultrasound in a plurality of directions with respect to a certain tissue, resulting in a superior image of the tissue. In addition, such a method can reduce noise, such as artifact present in the space, with respect to the signal.

In the above-described spatial compounding, in a two-dimensional compounding space, a plurality of sub-regions are generated; that is, a plurality of sub-regions are defined, based on a difference in the overlapping manner of a plurality of sets of frame data; that is, a difference in the overlapping structures. For example, when a basic frame data set of a rectangular shape obtained under the condition of the beam deflection angle of 0 degree, a positive-side deflection set of frame data of a parallelogram shape obtained under the condition of the beam deflection angle of +15 degrees, and a negative-side deflection set of frame data of a parallelogram shape obtained under the condition of the beam deflection angle of −15 degrees are compounded, there are generated a central sub-region (I) in which three sets of frame data overlap, two peripheral sub-regions (II) and (III) in which two sets of frame data overlap, and a deep sub-region (Iv) in which only the basic set of frame data exists. The deep sub-region (IV) is a region which is in general not displayed.

Basically, the spatial compounding executes an additive average process for coordinates in each region between a plurality of frames. In the above-described example configuration, in the central sub-region (I), an additive average calculation is executed to divide a sum of three echo values obtained from three sets of frame data by 3, and, in the two peripheral sub-regions (II) and (III), an additive average calculation is executed to divide the sum of two echo values obtained from two sets of frame data by 2.

However, when a case where the ultrasound beam is not deflected (case of the beam deflection angle of 0 degree) and a case where the ultrasound beam is deflected are compared, in general, a gain of the latter is lower than a gain of the former. This is because, even though the physical reception opening size on the array transducer is unchanged, the effective size of the reception opening is reduced with the increase in the beam deflection angle, and, similarly, the effective area of individual transducer element is reduced as the beam deflection angle is increased. An ultrasound propagation distance from the array transducer to an observation point at a certain depth also changes depending on the beam deflection angle, and, therefore, it is difficult to match the gains among a plurality of data sets having different deflection conditions, unless the transmission condition and the reception condition are precisely changed according to the beam deflection angle.

Under the circumstances described above, a gain difference tends to be generated between sub-regions in the compounded frame data after the compounding process, and there has been a problem in that, as a consequence, the boundary between the sub-regions stands out. Patent Document 1 discloses a technique which applies a weighted addition process in which the weight smoothly changes around the boundary between the sub-regions. Although such a process itself is effective, the gain difference between the sub-regions is not fundamentally resolved. Patent Document 2 also discloses a weighted addition process. Patent Document 2 also discloses that a variation of the signal amplitude is corrected. However, in order to precisely adjust the gain according to the beam deflection angle, very complex control would be required.

### RELATED ART REFERENCES

### Patent Documents

[Patent Document 1] JP 2003-61955 A
[Patent Document 2] JP 2002-526229 A

### DISCLOSURE OF INVENTION

### Technical Problem

As described, after the spatial compounding process, a gain difference tends to be generated between sub-regions. There has been a problem in that a boundary line which is an obstruction in the image tends to arise between the sub-regions on the ultrasound image due to such a gain difference. In particular, such a problem becomes particularly significant when the spatial compounding process is applied for harmonic components.

An advantage of the present invention is improvement of an image quality of an ultrasound image generated by the spatial compounding. Another advantage of the present invention is that the gain difference between sub-regions in the compounded framed data is not generated, or even if such a gain difference is generated, the gain difference is reduced. Yet another advantage of the present invention is that the gain difference is resolved or reduced by means of a simple structure.

### Solution to Problem

According to one aspect of the present invention, there is provided an ultrasound diagnostic apparatus, comprising: a transmitting and receiving unit that sequentially forms n beam scanning planes (where n is an integer greater than or equal to 2) while changing a beam deflection condition, to generate n sets of frame data; and a compounding unit that generates compounded frame data through a compounding process of the n sets of frame data, wherein a plurality of sub-regions are defined based on a difference in overlapping structures when the n sets of frame data are spatially overlapped, the plurality of sub-regions include a central sub-region in which all of the n sets of frame data are compounded and a plurality of peripheral sub-regions in which a portion of the n sets of frame data are compounded, and, in the compounding process in each of the peripheral sub-regions, a condition to compensate for insufficiency of a overlapping structure in the peripheral sub-region with respect to a overlapping structure in the central sub-region is applied, so that a gain after the compounding process in the central sub-region and a gain after the compounding process in each of the peripheral sub-regions are matched.

According to the above-described configuration, n beam scanning planes are formed by the transmitting and receiving unit. The n beam scanning planes are formed under beam deflection conditions that differ from each other. The compounding unit generates compounded frame data using the n sets of frame data corresponding to the n beam scanning planes. In the two-dimensional compounding space, a plurality of sub-regions are defined based on the difference in the manner of overlapping of the n sets of frame data; that is, the overlapping structures. In this case, theoretically, sub-regions having a number of overlaps of n to sub-regions having a number of overlaps of 1 would be generated. However, the sub-region or a portion which is outside of the display area may be omitted from the processing target. As a result of the actual overlapping structures being not uniform among the plurality of sub-regions, when a simple additive averaging process is executed in each sub-region, a gain difference would result. Therefore, in the above-described configuration, each sub-region appears to have the same overlapping structure. Desirably, the overlap structure insufficiency is compensated for in the compounding process so that a overlapping structure similar to that of the central sub-region (sub-region where all sets of frame data overlap) appears to be generated in the compounding process of each peripheral sub-region (sub-region where a portion of sets of frame data overlap). In this case, for example, frame data covering the peripheral sub-region may be repeatedly referred to, to increase in the appearance the data to be used in the compounding process. According to such a method, a common calculation equation for the compounding process may be employed among the plurality of sub-regions. Alternatively, in place of repeatedly referring to the same frame data, the data may be multiplied by a coefficient corresponding to the number of referrals, to obtain a similar result.

As described, the above-described method focuses on the difference in the actual overlapping structures among the sub-regions, and applies a necessary compensation (operation on the calculation), so that the overlapping structures appear to be the same among the sub-regions for the compounding process. In other words, the condition of the compounding process (desirably, an additive average condition) is matched among the plurality of sub-regions. With this configuration, the gain difference among the sub-regions is resolved or reduced. Such a configuration results in disappearance or reduction of the boundary line which appears on the ultrasound image and which is obstructive, and results in uniform feeling or brightness of the overall ultrasound image.

For the compensation of the insufficiency, ideally, there are used frame data having the same gain relationship as the missing frame data (having deflection angles symmetric about positive and negative signs). Alternatively, frame data having a close gain relationship (having deflection angles close to the deflection angles symmetric about the positive and negative signs) may be used. Desirably, the compounding process conditions of the peripheral sub-regions are matched with reference to the compounding process condition of the central sub-region. Alternatively, a compounding process condition of a particular peripheral sub-region may be set as a reference, and the compounding process conditions of the other sub-regions including the central sub-region may be matched to the reference.

Alternatively, in addition to the gain difference compensation as described above, the weighting process as described in Patent Document 1 may be applied simultaneously. According to such a combination, it becomes possible to set the boundary line to not stand out when the gain difference still remains even after the gain difference compensation as described above.

According to another aspect of the present invention, preferably, the condition to compensate for the insufficiency of the overlapping structure in the peripheral sub-region is a condition of referring to, in a duplicated manner, frame data which cover the peripheral sub-region and which have the same gain relationship as frame data which do not cover the peripheral sub-region. The same gain relationship is, in general, satisfied between a frame data pair obtained under beam deflection angles symmetric about positive and negative signs. Desirably, the plurality of beam deflection conditions are determined such that one or a plurality of such relationships are satisfied.

According to another aspect of the present invention, preferably, the number n is an odd number greater than or equal to 3, the n beam scanning planes include a beam scanning plane formed with a beam deflection angle of 0 degree, and at least one beam scanning plane pair formed with beam deflection angles symmetric about positive and negative signs, and a frame data pair corresponding to the beam scanning plane pair has the same gain relationship.

According to another aspect of the present invention, preferably, the compounding unit comprises: a compounding function which is a common function among the plurality of sub-regions, and that has the value n in a denominator and n additive elements in a numerator; and a correspondence table which is a table in which n frame data identifiers for giving n echo values to be substituted in the n additive elements are registered for each of the sub-regions, and in which the same frame data identifier is registered in a duplicated manner in order to compensate for the insufficiency of the overlapping structure for each of the peripheral sub-regions, and the compounding unit substitutes n echo values to the compounding function while referring to the correspondence table for each of the sub-regions, and executes the compounding function.

According to the above-described configuration, a common compounding function is employed among the plurality of sub-regions. In other words, it becomes no longer necessary to prepare a dedicated compounding function for each sub-region. The compounding function is desirably a function that applies an additive average process, and the denominator is the value n corresponding to the number of additions. The numerator is formed with n additive elements. The n additive elements are to be added, and actual values are substituted into the additive elements. The correspondence table instructs the substitution. In the table, the same frame data identifier is registered in a duplicated manner for a certain peripheral sub-region, so that the above-described compensation can be applied. The contents of the table do not need to be changed even when the beam deflection angle (absolute value) is changed, so long as the positive-negative symmetry is maintained. When the weighting process as described in the Patent Document 1 is to be applied in combination, the weight coefficient may be applied to the individual additive element of the numerator, or the weighting process may be applied to the echo values before the echo values are substituted in the compounding function.

According to another aspect of the present invention, preferably, the compounding unit further comprises an interpolation unit that generates all or a portion of the n echo values to be substituted into the n additive element by an interpolation process based on all or a portion of the n frame data. When a sampling arrangement (coordinate system) differs among the frame data, the necessary data are generated by an interpolation process based on the peripheral data.

According to another aspect of the present invention, preferably, the n sets of frame data are formed with harmonic echo components generated in a living body. Because the harmonic component is significantly attenuated as compared to the fundamental component, the difference in the gain due to the difference in the propagation distance or the like tends to be generated, and thus, application of the above-described method is desirable.

Alternatively, the compounding process of the plurality of sets of frame data can be executed by an information processing device such as a computer. In this case, a frame data array provided from an ultrasound system is input to the information processing device on-line or off-line.

### BRIEF DESCRIPTION OF DRAWINGS

FIG. 1 is a block diagram showing an ultrasound diagnostic apparatus according to a preferred embodiment of the present invention.

FIG. 2 is a conceptual diagram showing a first Example of a compounding process.

FIG. 3 is a diagram showing a first Comparative Example, and shows contents of a compounding process for each region.

FIG. 4 is a diagram showing a first Example, and shows contents of a compounding process for each region.

FIG. 5 is a diagram showing an example structure of the compounding unit shown in FIG. 1.

FIG. 6 is a conceptual diagram showing a second Example of the compounding process.

FIG. 7 is a diagram showing a second Comparative Example and shows contents of a compounding process for each region.

FIG. 8 is a diagram showing a second Example, and shows contents of a compounding process for each region.

### EMBODIMENT FOR CARRYING OUT THE INVENTION

A preferred embodiment of the present invention will now be described in detail with reference to the drawings.

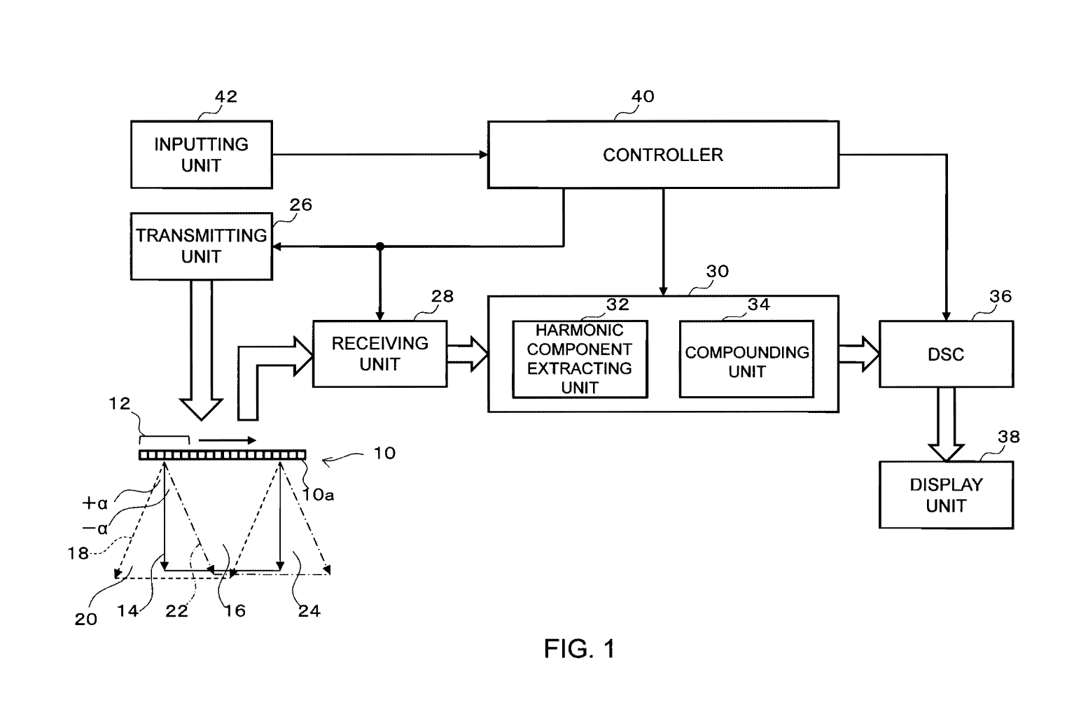

FIG. 1 shows an ultrasound diagnostic apparatus according to a preferred embodiment of the present invention. FIG. 1 is a block diagram schematically showing an overall structure of the ultrasound diagnostic apparatus. The ultrasound diagnostic apparatus is used in the medical field, and is a device which forms an ultrasound image by transmitting and receiving ultrasound to and from a living body.

In FIG. 1, an array transducer 10 is provided in a probe (not shown), and the ultrasound is transmitted and received by the array transducer 10. In the example configuration of FIG. 1, the array transducer 10 comprises a plurality of transducer elements 10a arranged in a linear shape. That is, in the present embodiment, a linear probe is employed. Alternatively, a convex type probe or the like may be employed.

An opening 12 is set on the array transducer 10. The opening 12 is, for example, a transmission and reception opening. By transmitting the ultrasound using the opening 12, a transmission beam is formed, and the reflected wave is received using the opening 12. Based on a plurality of reception signals generated by the reception, a reception beam is formed by a phase-alignment and summing process to be described later. Reference numeral 14 represents an example ultrasound beam, and the ultrasound beam 14 corresponds to a transmission/reception total beam (transmission/reception combined beam). The ultrasound beam 14 is formed in a direction perpendicular to the direction of arrangement of the array transducer 10. In other words, a beam deflection angle of the ultrasound beam 14 is 0 degree. By scanning the opening 12 along the arrangement direction; that is, by executing electric linear scanning, the ultrasound beam 14 is linearly scanned, and a scanning plane 16 is formed. The scanning plane 16 corresponds to a two-dimensional data capturing region. The scanning plane 16 has a rectangular shape.

In the present embodiment, in order to execute the spatial compounding method, scanning planes 20 and 24 are sequentially formed following the scanning plane 16. In other words, 3 scanning planes 16, 20, and 24 are generated in a circulating manner. More specifically, an ultrasound beam 18 is formed in a direction of the beam deflection angle of +α degrees on the positive side using the opening 12, and the ultrasound beam 18 is electric-scanned while the beam deflection angle is maintained, so that the scanning plane is formed. The scanning plane 20 can be considered a positive-side deflection scanning plane. Similarly, a beam deflection angle of −α degrees on the negative side is set using the opening 20, and an ultrasound beam 22 is formed in that direction. The ultrasound beam 22 is electric-scanned while the beam deflection angle is maintained, so that the scanning plane 24 is formed. The scanning plane 24 can be considered a negative-side deflection scanning plane. The scanning planes 20 and 24 have parallelogram shapes directed in opposite directions from each other.

In the present embodiment, the beam deflection angle of +α degrees which is set for forming the scanning plane 20 and the beam deflection angle of −α degrees which is set for forming the scanning plane 24 are the same angle (in terms of absolute value). That is, the scanning plane 20 and the scanning plane 24 form a pair. By setting the same beam deflection angle on both the positive side and the negative side, it becomes possible to match the gain. Similarly, in a case where a convex-type probe is used, a transmission and reception sequence similar to that described above may be applied.

A transmitting unit 26 is a transmission beam former, and supplies a plurality of transmission signals in parallel to the plurality of transducer elements during transmission. With this structure, ultrasound is transmitted to the inside of the living body. An echo reflected at each reflection point in the living body (reflected wave) is received by the array transducer 10, and a plurality of reception signals are output in parallel from the plurality of transducer elements forming the reception opening, and are input to a receiving unit 28. The receiving unit 28 is a reception beam former, and executes a phase-alignment and summing process for the plurality of received signals, to output beam data as a reception signal after the phase-alignment and summing.

One set of frame data (hereinafter simply referred to as a “frame”) is output for one scanning plane. One frame is formed with a plurality of sets of beam data arranged in the scanning direction, and each set of beam data is formed with a plurality of echo values (echo data) arranged in a depth direction. In reality, an echo data array in the time sequential order is transmitted.

A signal processor 30 is a module which executes various signal processes on the beam data. Alternatively, the signal processor 30 may be realized substantially by a function of software. The signal processor 30 generally comprises a wave detector, a gain controller, a logarithmic compressor, a frame correlation device, or the like. In the present embodiment, the signal processor 30 further comprises a harmonic component extractor 32 and a compounding unit 34. The harmonic component extractor 32 is a module for extracting a harmonic component generated in the living body. For example, the harmonic component included in the received signal can be extracted using various methods such as band-pass filtering, pulse inversion, pulse modulation, or the like. For example, when the pulse inversion is applied, two transmissions are executed while the phase is inverted for each beam address.

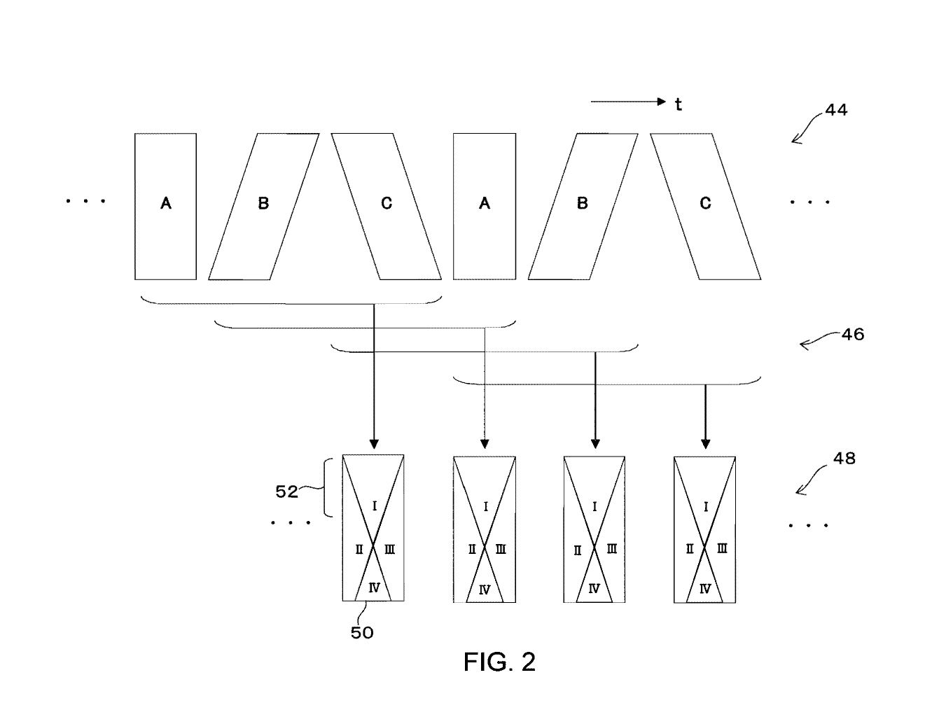

The compounding unit 34 is a module for executing the spatial compounding process. Specifically, in the present embodiment, in the frame array which is input in the time sequential order, a process to compound the frames, with 3 frames serving as a unit, to generate a compounded frame, is executed. The compounding unit 34 outputs a compounded frame array (compounded frame data array). A specific function of the compounding unit 34 will be described later in detail with reference to FIG. 2 or the like.

A digital scan converter (DSC) 36 is a module which forms the B-mode image (two-dimensional tomographic image) based on the input frames. The DSC 36 has an interpolation function, a coordinate conversion function, or the like. The generated image data are sent to a display unit 38 through a display processor (not shown). In the display unit 38, the B-mode image is displayed as a video image or a still image. In the present embodiment, a B-mode image to which the spatial compounding is applied; in particular, the B-mode image in which the gain difference within the frame is compensated for, can be displayed. Because of this, the image quality of the B-mode image can be improved as compared to the related art. When the extraction of the harmonic component as described above is executed, a harmonic image is displayed as the B-mode image. In this case, a harmonic generated in the tissue in the living body or a harmonic component generated at a contrast medium in the living body is displayed on a screen.

A controller 40 executes an operation control of the structures shown in FIG. 1. An inputting unit 42 is connected to the controller 40. The inputting unit 42 is formed with an operation panel, and the operation panel has various input devices such as a keyboard and a trackball. In the example structure of FIG. 1, for example, a cine memory is provided between the signal processor 30 and the DSC 36. The cine memory has a structure of a ring buffer storing a frame array. Alternatively, such a cine memory may be provided downstream of the DSC 36. In addition, the compounding unit 34 may be provided downstream of the DSC 36. In this case, the spatial compounding process is applied to a display frame array. In such a case also, preferably, the gain compensation condition to be described below is applied.

FIG. 2 shows a first configuration of a compounding process executed by the compounding unit shown in FIG. 1. Reference numeral 44 shows a frame array (frame data array) which is input to the compounding unit. The frame array 44 is made of a plurality of frames arranged in the time axis direction, and more specifically, is generally made of 3 types of frames A, B, and C. The frame A corresponds to the scanning plane 16 shown in FIG. 1; that is, the frame A is a frame obtained under the condition of the beam deflection angle of 0 degree. The frame B is a frame corresponding to the scanning plane 20 shown in FIG. 1; that is, a frame obtained under a condition of the beam deflection angle of +α degrees on the positive side. The frame C is a frame corresponding to the scanning plane 24 shown in FIG. 1; that is, a frame obtained under a condition of the beam deflection angle of −α degrees on the negative side. These 3 types of frames are obtained in a circulating manner.

In FIG. 2, reference numeral 46 shows a movement average process. In the movement average process 46, an additive average process is repeatedly executed on 3 frames of the frame array 44 as a unit. In FIG. 2, the units of the additive average process are shown with brackets. In this manner, the spatial compounding process; that is, a combining process, is sequentially executed on 3 frames a unit.

As a result of such a process, a compounded frame array (compounded frame data array) 48 is produced. The compounded frame array 48 is made of a plurality of compounded frames 50 arranged in the time sequential order. Each of the individual compounded frames 50 is produced by overlapping the frames A, B, and C on a two-dimensional compounding space, and portions running out on the right side and the left side are cut off. In other words, these portions are not the target of the compounding process. As a result, a region in the compounded frame 50 (overall region) is divided into 4 regions (sub-regions) based on differences in the manner of overlapping of the 3 frames; that is, differences in overlapping structures. More specifically, as shown in FIG. 2, a region I, a region II, a region III, and a region IV are defined.

Here, the region I is a region where all 3 of the frames A, B, and C are overlapped, the region II is a region where 2 frames, the frames A and B, are overlapped, the region III is a region where two regions, the frames A and C, are overlapped, and the region IV is a region covered only by the frame A. In other words, the number of overlaps in the region IV is 1. In this manner, based on the difference in the form of the 3 frames, the number of overlaps and the overlapping structure differ among the local regions in the compounded frame 50, and, as a result, 4 types of regions are defined. In reality, only a region near the probe is displayed. This region is shown in FIG. 2 as a display area 52. In reality, in principle, the region IV is not displayed. However, the size of the display area 52 in the depth direction; that is, the measurement range, can be varied and set by the user.

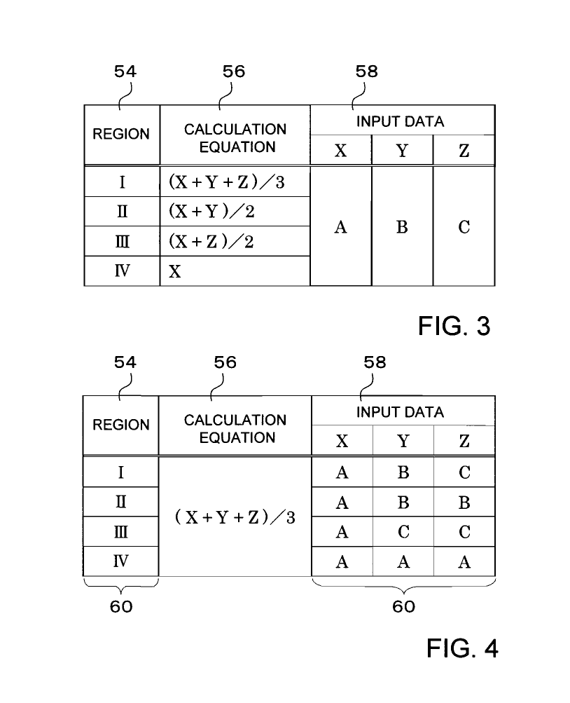

As described, because the overlapping structure or the number of overlaps differs depending on the partial region, if the additive averaging process is simply executed without taking such a difference into consideration, the above-described problem of the gain differences among the regions would be caused. This problem and the method of solving the problem will now be described with reference to FIGS. 3 and 4.

FIG. 3 shows a first Comparative Example. FIG. 3 is a diagram showing compounding process content for each region. FIGS. 4, 7, and 8 to be described later are also diagrams showing similar contents.

In FIG. 3, reference numeral 54 shows a region. Reference numeral 56 shows a calculation equation applied to each region. Reference numeral 58 shows input data to be given to additive elements X, Y, and Z included in the calculation equation. More specifically, identifiers A, B, and C of the frame type to which the echo values to be given to X, Y, and Z belong are shown. For example, in the region I, 3 echo values identified by A, B, and C are substituted into X, Y, and Z, a sum of these values is divided by the number of added elements; that is, 3, and the compounded echo data value is generated as an additive average value. The compounded echo data value forms one data value in the compounded frame, and, similarly, in the region II, an average of 2 echo values is calculated, and in region III, an average of 2 echo values is calculated. Here, it is to be noted that the denominator of the calculation equation used in these calculations is 2. In other words, for the beam formed in a direction orthogonal to the array transducer, in beams inclined from this beam, the gain is relatively reduced, and the gains are not matched among the frames A, B, and C. More specifically, the gain is lower for the frames B and C than for the frame A. Under such a circumstance, if the calculation equation for each region shown in FIG. 3 is to be applied, the gain becomes higher in the calculation result for the regions II and III than for the region I. In other words, in the regions II and III, because the ratio of the echo values having lower gains is increased in the numerator, the problem of the gain differences as described above is caused. Incidentally, the region IV has the largest gain.

FIG. 4 shows a first configuration in which the gain differences as described above is resolved. In the first configuration, the gain differences is resolved with a very simple structure. More specifically, as will be described below, a calculation equation common to the regions is used. This configuration will now be described in detail.

Similar to the above, reference numeral 54 shows a region, and reference numeral 56 shows the calculation equation. In the first configuration, as described above, a common calculation equation is used in a plurality of regions. In other words, only a single calculation equation is used. The calculation equation is D=(x+Y+z)/3. Here, D is the compounded echo value.

Such a single calculation equation can be used because a special operation is applied for compensating for the differences in the overlapping structures in the substitution of the 3 additive elements in the numerator. Specifically, with reference to the input data shown by reference numeral 58, 3 types of frames A, B, and C are correlated to the additive elements X, Y, and Z in the region I, which is identical to that of the Comparative Example shown in FIG. 3. However, in the first configuration, in the regions II and III, the same frame identifiers are given to the additive elements Y and Z; that is, the same frame is referred twice. In the calculation equation, the same echo value obtained from the same frame is substituted twice for the numerator.

In a further specific explanation, in the region II, as compared to the region I, the contribution from the frame C is missing, and the frame B is referred to in a duplicated manner in order to compensate for such a missing part. Similarly, in the region III, the contribution from the frame B is missing, and the frame C is referred to twice in order to compensate for the insufficiency. As a result, in all of the regions, a frame having a basic gain and two frames having a gain slightly lower than the basic frame are referred to, and the gain difference among the regions can be resolved. In the present embodiment, in the frame B and the frame C, beam deflection angles symmetric about the positive and negative signs around the beam deflection angle of 0 degree are applied, and, as a result, the frames B and C have a symmetric relationship or a pair relationship, and, thus, the duplicated reference as described above can be employed.

Alternatively, the calculation equation can be manipulated to give a coefficient for adjustment to the numerator for the region II or the region III, and to resolve the gain difference. Alternatively, the gains of the regions II and III may be set as a standard gain, and the gain of the region I may be reduced; more specifically, Y/2 may be applied in place of Y and Z/2 may be applied in place of Z, to resolve the gain difference.

According to the above-described first configuration, the single calculation equation can be used without further manipulation, to manipulate the echo value to be given to the calculation equation by a table shown by reference numeral 60, so that an advantage can be obtained in that the gain difference compensation can be realized with a simple structure. In other words, because the gain difference can be compensated for by merely registering the contents of the table 60, the structure is very simple. In particular, when the compounding unit shown in FIG. 1 is realized as a function of software, a combination of the single calculation equation and the table is preferably used.

In the first configuration, the frame B and the frame C satisfy a condition of the same deflection angle, but this does not need to be the case. More specifically, with a frame pair which does not have completely the same deflection angle, but satisfies a close angle condition, the gain difference can be reduced by applying the above-described method.

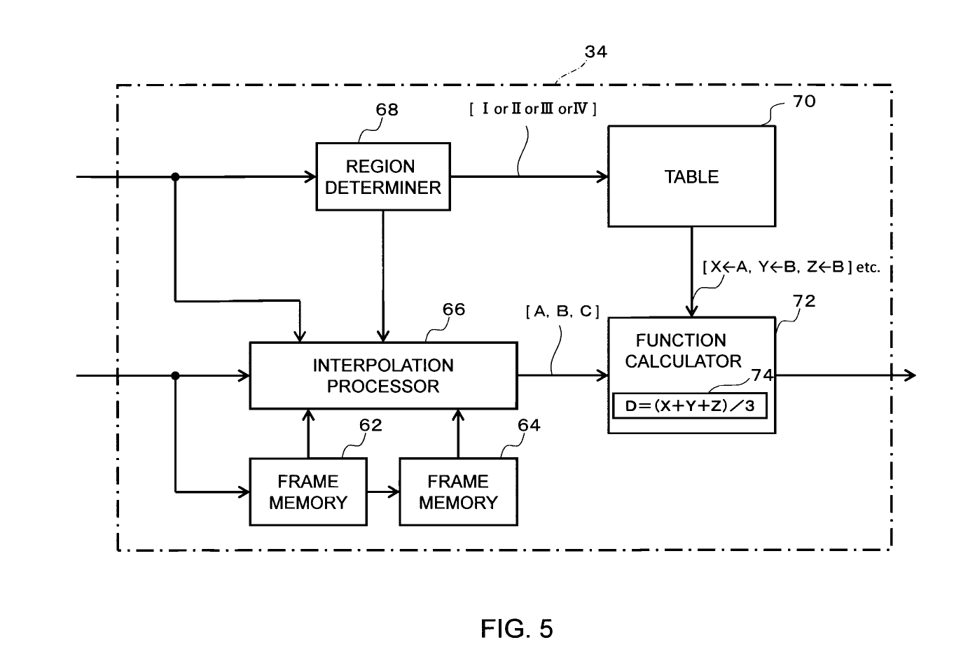

FIG. 5 shows as a block diagram an example structure of the compounding unit shown in FIG. 1. The compounding unit 34 is actually realized as a software function, and FIG. 5 shows such a function as a block diagram to facilitate understanding.

In FIG. 5, an interpolation processor 66 and a region determiner 68 are given address data in the example configuration that is shown. The address data shows an attribute of the echo value which is currently input; that is, the address data have information such as the type of the frame to which the echo value belongs, the coordinate in the frame, etc. On the other hand, the input echo value is input to the interpolation processor 66 and a frame memory 62. In other words, the frame array is input to the interpolation processor 66 and the frame memory 62. The frame memory 62 is a memory which stores a frame which is one frame prior to the current frame, and the frame which is output from the frame memory 62 is stored in a frame memory 64. The frame memory 64 is a memory which stores a frame which is two frames prior to the current frame. The current frame is input directly to the interpolation processor 66.

The region determiner 68 refers to the address data of an echo value which is currently input (echo value of interest), to determine a region to which the echo value belongs. That is, the region determiner 68 determines to which of the region I, region II, region III, and region IV the echo value of interest belongs. The determination result is output to a table 70. The table 70 is a table having the contents identified by the reference numeral 60 shown in FIG. 4. More specifically, the table 70 has the information for identifying the 3 frames for generating the 33 echo values to be given to the 3 additive elements of the numerator of the calculation equation. Information necessary for execution of the function calculation corresponding to the echo value of interest is output from the table 70. The information identifies the frame to which the echo values to be substituted into the additive elements belong. In the embodiment, the interpolation processor 66 is a module which executes the interpolation process in order to give the echo values to the coordinates of the frame A, with reference to the address space of the frame A, from the frames B and C. In other words, in the present embodiment, an interpolation process is applied on the frame B and frame C, to extract the echo value as an interpolated value. This is because the coordinate systems differ from each other among the 3 types of frames. Alternatively, the interpolation process may be applied to the frame A. In either way, when a calculation equation shown by reference numeral 74 is executed at a function calculator 72, the necessary echo value is given from the output of the table 70 to the function calculator 72, and the necessary echo value is given from the interpolation processor 66 to the function calculator 72. For example, for the region I, 3 echo values generated from the frames A, B, and C are substituted into the numerator of the calculation equation. In this case, the echo values generated from the frame B and frame C are interpolated values. In the regions II and III, the echo value extracted from the frame A, and the echo values which are interpolated values generated from the frame B or the frame C are substituted into the numerator of the calculation equation. In this case, as shown in FIG. 4, the same echo values are given in a duplicated manner as necessary for the additive elements Y and Z. With this process, the compounded echo value is generated using a common calculation equation while compensating for the insufficient echo value.

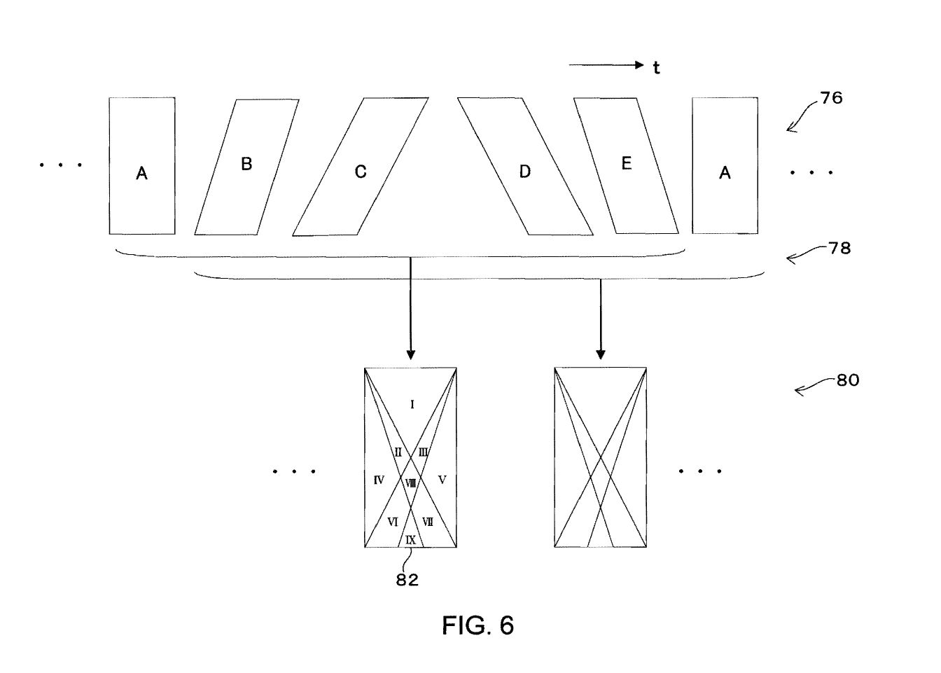

Next, a second configuration of the compounding process will be described with reference to FIGS. 6-8.

FIG. 6 is a conceptual diagram of the second configuration. Reference numeral 76 shows a frame array which is input to the compounding unit, and, in this example structure, 5 types of frames are input in a circulating manner. The 5 types of frames are identified in FIG. 6 as A, B, C, D, and E. The beam deflection conditions applied to the frames differ from each other; that is, the 5 types of frames are generated using 5 beam deflection angles. The frame A is a basic frame; that is, a frame generated under the condition of the beam deflection angle of 0 degree. The frames B and E are frames generated with the same deflection angle but different signs, and form a frame pair. Similarly, the frames C and D are a frame pair generated under the condition of symmetry in the positive and negative signs.

Reference numeral 78 shows the content of the movement average process. In the present embodiment, an additive average process is sequentially executed with 5 frames aligned on the time axis as a unit. Reference numeral 80 shows a compounded frame array, which is made of a plurality of compounded frames 82. Each compounded frame 82 is generated by overlapping the 5 types of frames; that is, through the compounding process. Nine types of regions are defined on the compounded frame 82 based on the difference in the overlapping state; that is, the difference in the overlapping structures. Thus, 9 regions from a region I to a region IX are present. In each region, the overlapping structure differs from the other regions, and, at the same time, the number of overlaps differs from the other regions.

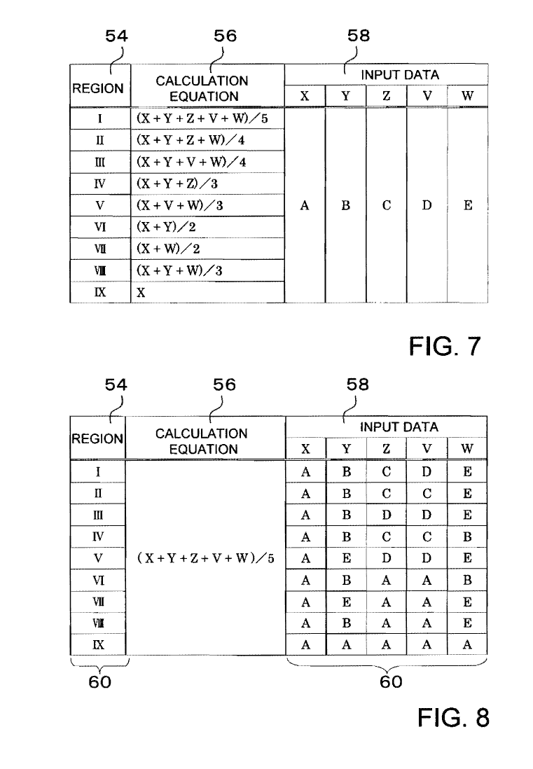

FIG. 7 shows a second Comparative Example. In a case where the compounding of the 5 types of frames shown in FIG. 6 is to be executed, if the compounding process is executed under the condition shown in FIG. 7, as described above, a gain difference would be caused among the regions. In other words, the calculation equations differ among the regions; in particular, the denominator differs among the regions, and calculation equations which differ from each other must be prepared for the regions. In addition, because a simple additive average process is executed in each region, there is a problem in that the gain difference according to the type of the frame would also appear in the compounded result.

FIG. 8 shows a second configuration that solves the above-described problem. As shown in FIG. 8, a common calculation equation is set for the 9 regions, and the number of overlaps of 5 corresponding to the region I (that is, the basic region or the central region) is shown in the denominator. The numerator of the calculation equation is made of 5 additive elements; that is, X, Y, Z, V, and W. For each additive element, input data is correlated as shown by reference numeral 58. In the drawing, A-E show the frame types; that is, the frame identifiers. An echo value is referred from an identified frame, and the echo value is substituted into the corresponding additive element.

For example, in the region II, the frame C is referred twice, and the compensation for the missing frame is realized by the duplicated registration. This is similarly applicable for the region III, in which the frame D is registered in a duplicated manner. In the region IV, with reference to the overlapping structure of the region I, the frame D and the frame E are each referred to twice in order to match the overlapping structure of the region I. In the other regions also, the duplicated registration for matching the overlapping structure of the region I is executed, and the gains are matched among the regions with this process.

Incidentally, in the region IX, the frame A is correlated with 5 additive elements, and the gain would become high. However, the region IX is the deepest region, and is a portion which is actually not displayed. Thus, no problem is caused by this configuration.

As described, in the second configuration also, the attributes of the echo values given to the additive elements are managed such that an overlapping structure similar to the basic region is generated. In other words, the necessary duplicated registration is applied. With this process, the gain difference among the regions can be resolved or reduced, and, thus, the quality of the ultrasound image can be significantly improved.

In the above-described embodiment, when it is necessary to dim the boundary between the regions more, a weighted addition is preferably applied with a smooth weight change around the boundary. In this case, in the calculation equations shown in FIGS. 4 and 8, each additive element can be multiplied with the weight coefficient, and the weight coefficient can be changed, so that the weighting process is realized simultaneously with the gain compensation described above.

In the above-described embodiments, the compounding process is executed with 3 or 5 frame sets including the reference frame as a unit, but alternatively, the compounding process may be applied with an even number of frames as a unit. In addition, although it is desirable that the number of types of the scanning planes and the number of frames used for the movement average process coincide, these numbers may differ from each other.

1.-6. (canceled)

7. An ultrasound diagnostic apparatus, comprising:
a transmitting and receiving unit that sequentially forms n beam scanning planes (where n is an integer greater than or equal to 2) while switching a beam deflection condition, to generate n sets of frame data; and
a compounding unit that generates compounded frame data through a compounding process of the n sets of frame data, wherein
a plurality of sub-regions are defined based on a difference in overlapping structures when the n sets of frame data are spatially overlapped,
the plurality of sub-regions include a central sub-region in which all of the n sets of frame data are compounded and a plurality of peripheral sub-regions in which a portion of the n sets of frame data are compounded,
in the compounding process in each of the peripheral sub-regions, a condition to compensate for insufficiency of a overlapping structure in the peripheral sub-region with respect to a overlapping structure in the central sub-region is applied, so that a gain after the compounding process in the central sub-region and again after the compounding process in each of the peripheral sub-regions are matched, and
in the compounding process in each of the peripheral sub-regions, the insufficiency of the overlapping structure in the peripheral sub-region with respect to the overlapping structure in the central sub-region is compensated for by a duplicated use of the same frame data which cover the peripheral sub-region.

8. The ultrasound diagnostic apparatus according to claim 7, wherein
in the compounding process in each of the peripheral sub-regions, the same frame data which cover the peripheral sub-region are referred twice.

9. The ultrasound diagnostic apparatus according to claim 7, wherein
the number n is an odd number greater than or equal to 3,
the n beam scanning planes include a beam scanning plane formed with a beam deflection angle of 0 degree, and at least one beam scanning plane pair formed with beam deflection angles symmetric about positive and negative signs, and
a frame data pair corresponding to the beam scanning plane pair has the same gain relationship.

10. The ultrasound diagnostic apparatus according to claim 7, wherein
the compounding unit comprises:
a compounding function which is a common function among the plurality of sub-regions, and that has the value n in a denominator and n additive elements in a numerator; and
a correspondence table which is a table in which n frame data identifiers for giving n echo values to be substituted for the n additive elements are registered for each of the sub-regions, and in which the same frame data identifier is registered in a duplicated manner in order to compensate for the insufficiency of the overlapping structure for each of the peripheral sub-regions, and
the compounding unit substitutes n echo values to the compounding function while referring to the correspondence table for each of the sub-regions, and executes the compounding function.

11. The ultrasound diagnostic apparatus according to claim 10, wherein
the compounding unit further comprises an interpolation unit that generates all or a portion of the n echo values to be substituted into the n additive elements by an interpolation process based on all or a portion of the n sets of frame data.

12. The ultrasound diagnostic apparatus according to claim 7, wherein
the n sets of frame data are formed with harmonic echo components generated in a living body.
